(19) United States
(12) Patent Application Publication
(10) Pub. No.: US 2014/0378702 A1
(43) Pub. Date: Dec. 25, 2014

(54) Processes for Separating Crude Acrylic Acids and Acrylates Comprising A Michael Addition Product

(71) Applicant: Celanese International Corporation, Irving, TX (US)

(72) Inventors: Craig J. Peterson, Houston, TX (US); Elizabeth Bowden, Houston, TX (US); Josefina T. Chapman, Houston, TX (US); Sean Mueller, Pasadena, TX (US)

(21) Appl. No.: 13/926,362

(22) Filed: Jun. 25, 2013

(51) Int. Cl. C07B 63/02; C07C 51/487; C07C 51/42

(52) U.S. Cl. CPC C07B 63/02; C07C 51/42; C07C 51/487
USPC 562/600

(57) ABSTRACT

In one embodiment, the present invention is directed to a process for producing an acrylate product. The process comprises the step of reacting, optionally in a reaction zone, a reaction mixture comprising an alkanoic acid and an alkylenating agent under conditions effective to form a crude acrylate product. The crude acrylate product comprises acrylate product and acetic acid. The process further comprises the step of reacting, in a separation zone, at least a portion of the acrylate product and at least a portion of the acetic acid to form an intermediate acrylate product. The intermediate acrylate product comprises a Michael addition by-product, e.g., 3-acetoxypropionic acid and/or 3-acetoxypropanoic acid. The process further comprises the step of separating at least a portion of the intermediate acrylate product to form a by-product stream and a purified acrylate product stream.

### FIELD OF THE INVENTION

The present invention relates generally to the production of acrylate product via the aldol condensation of an alkanoic acid. More specifically, the present invention relates to a Michael addition by-product that may be formed during the acrylate product separation process.

### BACKGROUND OF THE INVENTION

α,β-unsaturated acids, particularly acrylic acid and methacrylic acid, and the ester derivatives thereof are useful organic compounds in the chemical industry. These acids and esters are known to readily polymerize or co-polymerize to form homopolymers or copolymers. Often the polymerized acids are useful in applications such as superabsorbents, dispersants, flocculants, and thickeners. The polymerized ester derivatives are used in coatings (including latex paints), textiles, adhesives, plastics, fibers, and synthetic resins.

Because acrylic acid and its esters have long been valued commercially, many methods of production have been developed. One exemplary acrylic acid ester production process utilizes: (1) the reaction of acetylene with water and carbon monoxide; and/or (2) the reaction of an alcohol and carbon monoxide, in the presence of an acid, e.g., hydrochloric acid, and nickel tetracarbonyl, to yield a crude product comprising the acrylate ester as well as hydrogen and nickel chloride. Another conventional process involves the reaction of ketene (often obtained by the pyrolysis of acetone or acetic acid) with formaldehyde, which yields a crude product comprising acrylic acid and either water (when acetic acid is used as a pyrolysis reactant) or methane (when acetone is used as a pyrolysis reactant). These processes have become obsolete for economic, environmental, or other reasons.

More recent acrylic acid production processes have relied on the gas phase oxidation of propylene, via acrolein, to form acrylic acid. The reaction can be carried out in single- or two-step processes but the latter is favored because of higher yields. The oxidation of propylene produces acrolein, acrylic acid, acetaldehyde and carbon oxides. Acrylic acid from the primary oxidation can be recovered while the acrolein is fed to a second step to yield the crude acrylic acid product, which comprises acrylic acid, water, small amounts of acetic acid, as well as impurities such as furfural, acrolein, and propionic acid. Purification of the crude product may be carried out by azeotropic distillation. Although this process may show some improvement over earlier processes, this process suffers from production and/or separation inefficiencies. In addition, this oxidation reaction is highly exothermic and, as such, creates an explosion risk. As a result, more expensive reactor design and metallurgy are required. Also, the cost of propylene is often prohibitive.

The aldol condensation reaction of formaldehyde and acetic acid and/or carboxylic acid esters has been disclosed in literature. This reaction forms acrylic acid and is often conducted over a catalyst. For example, condensation catalysts consisting of mixed oxides of vanadium and phosphorus were investigated and described in M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987).

Processes for preparing acrylic acid from methanol and acetic acid or from ethanol and formaldehyde have been disclosed in U.S. Pat. Pubs. 2012/0071688 and 2012/0071687.

Even in view of these references, the need exists for a process for producing purified acrylate product, which addresses the Michael addition by-product that may potentially be formed in the reaction zone and/or the separation zone. In particular, the need exists for a process capable of removing the Michael addition by-product, thus formed, to yield the purified acrylate product.

The references mentioned above are hereby incorporated by reference.

### BRIEF DESCRIPTION OF DRAWINGS

The invention is described in detail below with reference to the appended drawings, wherein like numerals designate similar parts.

FIG. 1 is a process flowsheet showing an acrylic acid reaction/separation system in accordance with an embodiment of the present invention.

FIG. 2 is a schematic diagram of an acrylic acid reaction/separation system in accordance with one embodiment of the present invention.

### SUMMARY OF THE INVENTION

In one embodiment, the present invention is directed to a process for producing an acrylate product. The process comprises the step of reacting, optionally in a reaction zone, a reaction mixture comprising an alkanoic acid and an alkylenating agent under conditions effective to form a crude acrylate product. The crude acrylate product comprises acrylate product and acetic acid. The process further comprises the step of reacting, in a separation zone, at least a portion of the acrylate product and at least a portion of the acetic acid to form an intermediate acrylate product. The reaction may be conducted at a temperature greater than 50° C. The intermediate acrylate product comprises a Michael addition by-product, e.g., 3-acetoxypropionic acid and/or 3-acetoxypropanoic acid. The process further comprises the step of separating at least a portion of the intermediate acrylate product to form a by-product stream and a purified acrylate product stream. The by-product stream comprises Michael addition by-product and the purified acrylate product stream comprises acrylate product. In one embodiment, the process may further comprise the step of directing at least a portion of the by-product stream to the reaction zone. In one embodiment, the process may further comprise the step of reacting, e.g., decomposing at least a portion of the Michael addition by-product in the by-product stream to form acrylic acid and acetic acid and, optionally, the step of recycling the acrylic acid and/or the acetic acid to the reaction zone and/or another component of the separation zone. The reaction zone may comprise a decomposition catalyst, which may be selected from the group consisting of acid catalysts, e.g., sulfuric acid, heteropolyacids, gel ion exchange resins, sulfonated resins, sulfated and phosphated metal oxides, supported heteropolyacids, metal sulfates, metal phosphates, aluminosilcates, zeolites, mixed metal oxides, and mixtures thereof. The separation zone may comprise at least one column and step (b) may be conducted in the at least one column. For example, the separation zone may comprise a first column and a second column and step (b) may be conducted in the first column. The by-product stream may exit the first column. The process may further comprise the steps of reacting at least a portion of the Michael addition by-product in the by-product stream to form recovered acrylate product and recovered alkanoic acid and/or directing at least a portion of the recovered acrylate product and alkanoic acid to a separation unit other than the first column, e.g., the second column. The process may comprise the step of directing at least a portion of the recovered acrylic acid and acetic acid to the first column. The purified acrylate product stream may comprise less than 25 wt % Michael addition by-product. The by-product stream may comprise at least 0.001 wt % Michael addition product. In another aspect, the invention may relate to a process for producing an acrylate product composition comprising acrylate product and Michael addition product comprising the steps of reacting alkylenating agent and alkanoic acid to form a crude acrylate product and heating the crude acrylate product to form the acrylate product composition comprising Michael addition product.

### DETAILED DESCRIPTION OF THE INVENTION

### Introduction

Production of unsaturated carboxylic acids such as acrylic acid and methacrylic acid and the ester derivatives thereof via most conventional processes have been limited by economic and environmental constraints. In the interest of finding a new reaction path, the aldol condensation reaction of acetic acid and an alkylenating agent, e.g., formaldehyde, has been investigated. This reaction may yield a unique crude acrylate product that comprises, inter alia, acrylate product, alkanoic acid, and a higher amount of (residual) formaldehyde, which is generally known to add unpredictability and problems to separation schemes. Although there may be some disclosure relating to the aldol condensation reaction, there is little if any disclosure relating to suitable separation schemes for handling the separation of the high formaldehyde content crude products. In particular, there is little if any disclosure relating to a reaction, e.g., a Michael addition reaction, of some of the components of the crude product to form a Michael addition by-product. Nor is there disclosure relating to the subsequent separation of the Michael addition by-product, thus formed, to form the purified acrylate product.

It has now been discovered that some components of the crude acrylate product formed via the aldol condensation reaction may, under some conditions, be reacted to form (or may react to form) by-products. Specifically, acrylate product, e.g., acrylic acid, and alkanoic acid, e.g., acetic acid, may undergo, under some conditions, a Michael addition reaction to form a Michael addition by-product. Without being bound by theory, it is believed that some components of the crude acrylate product stream may react with one another when the crude acrylate product is separated in the separation zone and/or when the components are heated. An intermediate acrylate product may be thus formed. As one example, the Michael addition reaction may occur in the separation zone. The separation zone may comprise at least one separation unit, e.g., a separation column. In one embodiment, the reaction may occur (at least minimally) when the two reactants are combined. In one embodiment, the reaction may occur when the crude acrylate product components are heated, e.g., heated to a temperature greater than 25° C., greater than 50° C., greater than 75° C., or greater than 100° C. The heating of the components, in some cases, may induce or increase the rate of the Michael addition reaction, as compared to mere combination of the reactants. In one embodiment, the invention may relate to a process for producing an acrylate product composition comprising acrylate product and Michael addition product. The process may comprise the steps of reacting alkylenating agent and alkanoic acid to form a crude acrylate product and heating the crude acrylate product to form the (intermediate) acrylate product composition comprising Michael addition product. As another example, the Michael addition reaction may occur when the components remain in the separation unit for a prolonged period of time, e.g., when residence time is greater than 1 minute, e.g., greater than 3 minutes, greater than 10 minutes, greater than 30 minutes, greater than 1 hour, greater than 2 hours, or greater than 5 hours.

In one embodiment, the separation zone comprises a first column and a second column. Preferably, the Michael addition reaction is conducted (at least) in the first column. The Michael addition by-product may exit the first column.

In cases in which Michael addition by-product is formed, the formation drives down the overall efficiency of the production process. By further processing the by-products as discussed herein, these inefficiencies may be reduced or eliminated. As one example of inefficiency, the acrylic acid that is reacted to form the Michael addition by-product is not recovered and, as such, overall yield is decreased. As another example, the acetic acid that is reacted to form the Michael addition by-product is lost and cannot be subsequently separated and/or recycled to the reaction zone for further reaction. It has now been discovered that the Michael addition product may be reacted, e.g., decomposed or cracked, to yield acrylate product and/or alkanoic acid. The acrylate product and/or alkanoic acid, thus formed, may then be further treated or otherwise utilized in the process. For example, the acrylate product, e.g., acrylic acid, may be purified and collected as purified acrylate product. As another example, the alkanoic acid, e.g., acetic acid, may be separated and/or recycled to the reaction zone for further reaction. By cracking the Michael addition by-product that was formed, e.g., during separation, to yield acrylate product and alkanoic acid, production efficiencies are advantageously improved, as compared to a conventional process in which the formed Michael addition product goes unutilized.

Accordingly, in one embodiment, the present invention relates to a process for producing an acrylate product and comprises the step of reacting a reaction mixture comprising an alkanoic acid and an alkylenating agent, optionally in a reactor, under conditions effective to form a crude acrylate product. The crude acrylate product comprises inter alia acrylate product, e.g., acrylic acid, and (residual) alkanoic acid, e.g., acetic acid.

As used herein, acrylic acid, methacrylic acid, and/or the salts and esters thereof, collectively or individually, may be referred to as “acrylate products.” The use of the terms acrylic acid, methacrylic acid, or the salts and esters thereof, individually, does not exclude the other acrylate products, and the use of the term acrylate product does not require the presence of acrylic acid, methacrylic acid, and the salts and esters thereof.

At least some of the components of the crude acrylate product, e.g., the acrylate product and the alkanoic acid, may then be reacted to form an intermediate acrylate product comprising, inter alia, a by-product. Preferably, the reaction is a Michael addition reaction and the resultant by-product is a Michael addition by-product. Michael addition may be referred to as the nucleophilic addition of a carbanion or another nucleophile (the Michael donor) to an α,β-unsaturated carbonyl compound (the Michael acceptor). In one embodiment, the Michael donor is an acetic acid anion and the Michael acceptor is acrylic acid. In one embodiment, an acrylic acid anion and acrylic acid are reacted to form a Michael addition product, e.g., 3-acetoxypropionic acid. The Michael addition by-product may comprise one or more Michael addition products. Preferably, the Michael addition product comprises 3-acetoxypropanoic acid and/or 3-acetoxypropionic acid. Other exemplary Michael addition products include the reaction products of propionic acid with additional propionic acid and the reaction products of propionic acid with acrylic acid and/or acetic acid.

The intermediate acrylate product comprises by-product. In one embodiment, the intermediate acrylate product comprises greater than 0.01 wt % by-product, e.g., greater than 0.012 wt %, greater than 0.015 wt %, greater than 0.025 wt %, greater than 0.1 wt %, greater than 0.5 wt %, greater than 0.65 wt %, greater than 0.75 wt % or greater than 1 wt %. In terms of ranges, the intermediate acrylate product may comprise from 0.01 wt % to 40 wt % by-product, e.g., from 0.01 wt % to 25 wt %, from 0.012 wt % to 25 wt %, from 0.012 wt % to 10 wt %, from 0.015 wt % to 10 wt %, from 0.1 wt % to 10 wt %, from 0.5 wt % to 10 wt %, from 0.65 wt % to 40 wt %, or from 0.65 wt % to 10 wt %. In terms of upper limits, the intermediate acrylate product may comprise less than 40 wt % by-product, e.g., less than 25 wt %, less than 10 wt %, or less than 5 wt %. Exemplary ranges for some components of the intermediate acrylate product stream are shown in Table 1. Components other than those listed in Table 1 may also be present in the intermediate acrylate product stream, e.g., methanol, methyl acetate, methyl acrylate, dimethyl ketone, carbon dioxide, carbon monoxide, oxygen, nitrogen, and acetone.

TABLE 1
INTERMEDIATE ACRYLATE PRODUCT
STREAM COMPOSITION

| | Conc. (wt. %) | Conc. (wt. %) | Conc. (wt. %) |
|---|---|---|---|
| Acrylic Acid | at least 5 | 5 to 99 | 35 to 65 |
| Acetic Acid | less than 95 | 5 to 90 | 20 to 60 |
| By-Product | 0.01 to 40 | 0.5 to 15 | 1 to 10 |
| Water | less than 25 | 0.1 to 10 | 0.5 to 7 |
| Alkylenating Agent | <1 | <0.5 | <0.1 |
| Propionic Acid | <10 | 0.01 to 5 | 0.01 to 1 |

The reaction may be conducted at various positions in the process. In one embodiment, the reaction occurs after the crude product exits the reactor. In one embodiment, the reaction occurs as the crude acrylate product is separated, e.g., the reaction occurs in the separation zone. As one example, the reaction may occur in at least one separation unit of the separation zone. Without being bound by theory, it is believed that some of the conditions of the separation zone induce the reaction of the acrylate product and the alkanoic acid. In one embodiment, the reaction may occur when the components reach a certain temperature, e.g., the temperatures discussed herein.

The process, in one embodiment, further comprises the step of separating at least a portion of the intermediate acrylate product to form a by-product stream and a purified acrylate product stream. At least a portion of the intermediate acrylate product may, in some embodiments, include derivatives of the intermediate acrylate product, e.g., streams derived via the separation of the intermediate acrylate product. In some cases, derivatives of the intermediate acrylate product may be separated to form the by-product stream. In one embodiment the Michael addition reaction and the separation occur in the same unit. The by-product stream comprises Michael addition by-product and the purified acrylate product stream comprises acrylate product. In one embodiment, the by-product stream comprises at least 0.001 wt % Michael addition product, based on the total weight of the by-product stream, e.g., at least 0.01 wt %, at least 0.1 wt %, at least 0.5 wt %, at least 1 wt %, at least 5 wt %, at least 10 wt %, or at least 25 wt %. In terms of ranges, the by-product stream may comprise from 0.001 wt % to 75 wt % Michael addition product, e.g., from 0.01 to 50 wt %, from 0.1 wt % to 50 wt %, or from 0.1 wt % to 5 wt %. Thus, at least a portion of the Michael addition by-product that is formed during separation is subsequently separated from the acrylate product. As a result the purified acrylate product is formed. The purified acrylate product, beneficially, comprises little if any Michael addition by-product. For example, the purified acrylate product may comprise less than 25 wt % Michael addition by-product, e.g., less than 10 wt %, less than 5 wt %, less than 1 wt %, less than 0.5 wt %, less than 0.1 wt %, or less than 0.05 wt %. Acrylate product formed via conventional separation schemes that do not take into account the formation of Michael addition by-products may be much less pure and may contain significantly higher amounts of Michael addition by-product. Weight percentages may be based on the total weight of the purified acrylate product.

The separated Michael addition by-product may be further treated or otherwise utilized. In one embodiment, at least a portion of the by-product stream, e.g., the Michael addition by-product, may be directed back to the reaction zone, e.g., for further reaction. In one embodiment, the separation zone comprises a first column and a second column. Preferably, the Michael addition reaction and the subsequent separation thereof are conducted in the first column. The Michael addition by-product may exit a column of the separation zone, e.g., the first column, and may be directed elsewhere in the process.

In one embodiment, the process comprises the step of reacting at least a portion of the Michael addition by-product in the by-product stream to form (recovered) acrylate product and alkanoic acid. This reaction, in some embodiments, may be a decomposition or cracking reaction. Thus, in some cases, the Michael addition product is cracked or decomposed into the components that were initially reacted, optionally under certain conditions, to form the Michael addition by-product. Preferably this reaction is conducted over a catalyst, e.g., a decomposition or cracking catalyst. The reaction may be conducted at various positions in the process. For example, this reaction may be conducted in the reaction zone. In such cases, the reaction zone may comprise one or more reactor that contains the decomposition or cracking catalyst. In one embodiment, the reactor that is utilized for the aldol condensation reaction may also be utilized for the decomposition reaction. In these instances, the reactor may be charged with both the aldol condensation catalyst and the decomposition/cracking catalyst. The reaction may also be conducted at various points in the separation zone. As one example, the reaction may be conducted at a point after which acrylate product, e.g., acrylic acid, and alkanoic acid, e.g., acetic acid, are separated from one another. In these cases, the separated acrylate product may comprise some remaining alkanoic acid and/or the separated alkanoic acid may comprise some remaining acrylate product.

The decomposition/cracking catalyst may vary widely. In one embodiment, the decomposition/cracking catalyst may be selected from the group consisting of acids, e.g., homogeneous strong acids such as sulfuric, hydrochloric, toluenesulfonic acid, trifluoromethanesulfonic acid, heteropolyacids such as salts of H₃PW₁₂O₄₀; heterogeneous solid acids such as macroreticular or gel ion exchange resins, sulfonated resins, Nafion®, sulfated or phosphated metal oxides such as sulfated zirconia; supported heteropolyacids; metal sulfates; phosphates such as AlPO₄ and NbPO₄; aluminosilcates; zeolites; mixed metal oxides; and combinations thereof. In a preferred embodiment, the decomposition/cracking catalyst is an acid catalyst, e.g., sulfuric acid.

Once the Michael addition by-product has been decomposed or cracked into the initial components, these recovered components can be utilized elsewhere in the process. As such, the process, in one embodiment, comprises the step of directing at least a portion of the by-product stream, e.g., at least some of the components of the by-product stream, to the reaction zone and/or to a different point in the separation zone. In some embodiments, the separation zone comprises a first column and at least one additional column. At least a portion of the acrylate product and/or the alkanoic acid generated by the decomposition reaction may be directed to a column other than the first column. In some embodiments, the separation zone comprises a first column and a second column. At least a portion of the acrylate product and/or the alkanoic acid generated by the decomposition reaction may be directed to a second column. In one embodiment, the process comprises the step of directing at least a portion of the acrylate product and alkanoic acid generated by the decomposition reaction to the first column. In one embodiment, the recovered products, e.g., acetic acid, may be directed to the reactor, where the acetic acid can be utilized as a reactant, which, beneficially, improves reaction overall process efficiency. In one embodiment, the recovered products, e.g., acrylic acid, may be directed to a downstream portion of the separation zone for further purification. Preferably, at least a portion of the recovered acrylate product and alkanoic acid may be fed to a column used to separate acrylate product from alknaoic acid. In doing so, the recovered acrylic acid, advantageously, is captured as usable product, as opposed to being lost as Michael addition by-product.

In addition to the components discussed above, in one embodiment, the crude acrylate product of the present invention further comprises water. For example, the crude acrylate product may comprise less than 60 wt. % water, e.g., less than 50 wt. %, less than 40 wt. %, or less than 30 wt. %. In terms of ranges, the crude acrylate product may comprise from 1 wt. % to 60 wt. % water, e.g., from 5 wt. % to 50 wt. %, from 10 wt. % to 40 wt. %, or from 15 wt. % to 40 wt. %. In terms of upper limits, the crude acrylate product may comprise at least 1 wt. % water, e.g., at least 5 wt. %, at least 10 wt. %, or at least 15 wt. %.

The crude acrylate product of the present invention comprises very little, if any, of the impurities found in most conventional acrylic acid crude acrylate products. For example, the crude acrylate product of the present invention may comprise less than 1000 wppm of such impurities (either as individual components or collectively), e.g., less than 500 wppm, less than 100 wppm, less than 50 wppm, or less than 10 wppm. Exemplary impurities include acetylene, ketene, beta-propiolactone, higher alcohols, e.g., C₂₊, C₃₊, or C₄₊, and combinations thereof. Importantly, the crude acrylate product of the present invention comprises very little, if any, furfural and/or acrolein. In one embodiment, the crude acrylate product comprises substantially no furfural and/or acrolein, e.g., no furfural and/or acrolein. In one embodiment, the crude acrylate product comprises less than less than 500 wppm acrolein, e.g., less than 100 wppm, less than 50 wppm, or less than 10 wppm. In one embodiment, the crude acrylate product comprises less than less than 500 wppm furfural, e.g., less than 100 wppm, less than 50 wppm, or less than 10 wppm. Furfural and acrolein are known to act as detrimental chain terminators in acrylic acid polymerization reactions. Also, furfural and/or acrolein are known to have adverse effects on the color of purified product and/or to subsequent polymerized products.

In addition to the acrylic acid and the alkylenating agent, the crude acrylate product may further comprise acetic acid, water, propionic acid, and light ends such as oxygen, nitrogen, carbon monoxide, carbon dioxide, methanol, methyl acetate, methyl acrylate, acetaldehyde, hydrogen, and acetone. Exemplary compositional data for the crude acrylate product are shown in Table 2. Components other than those listed in Table 2 may also be present in the crude acrylate product.

TABLE 2
CRUDE ACRYLATE PRODUCT STREAM COMPOSITIONS

| | Conc. | Conc. | Conc. | Conc. |
|---|---|---|---|---|
| Component | (wt. %) | (wt. %) | (wt. %) | (wt. %) |
| Acrylic Acid | 1 to 75 | 1 to 50 | 5 to 50 | 10 to 40 |
| Alkylenating Agent(s) | 0.5 to 50 | 1 to 45 | 1 to 25 | 1 to 10 |
| Acetic Acid | 1 to 90 | 1 to 70 | 5 to 50 | 10 to 50 |
| Water | 1 to 60 | 5 to 50 | 10 to 40 | 15 to 40 |
| Propionic Acid | 0.01 to 10 | 0.1 to 10 | 0.1 to 5 | 0.1 to 1 |
| Oxygen | 0.01 to 10 | 0.1 to 10 | 0.1 to 5 | 0.1 to 1 |
| Nitrogen | 0.1 to 20 | 0.1 to 10 | 0.5 to 5 | 0.5 to 4 |
| Carbon Monoxide | 0.01 to 10 | 0.1 to 10 | 0.1 to 5 | 0.5 to 3 |
| Carbon Dioxide | 0.01 to 10 | 0.1 to 10 | 0.1 to 5 | 0.5 to 3 |
| Other Light Ends | 0.01 to 10 | 0.1 to 10 | 0.1 to 5 | 0.5 to 3 |

### Production of Acrylate Products

Any suitable reaction and/or separation scheme may be employed to form the crude acrylate product as long as the reaction provides the crude acrylate product components that are discussed above. For example, in some embodiments, the acrylate product stream is formed by contacting an alkanoic acid, e.g., acetic acid, or an ester thereof with an alkylenating agent, e.g., a methylenating agent, for example formaldehyde, under conditions effective to form the crude acrylate product stream. Preferably, the contacting is performed over a suitable catalyst. The crude acrylate product may be the reaction product of the alkanoic acid-alkylenating agent reaction. In a preferred embodiment, the crude acrylate product is the reaction product of the aldol condensation reaction of acetic acid and formaldehyde, which is conducted over a catalyst comprising vanadium and titanium. In one embodiment, the crude acrylate product is the product of a reaction in wherein methanol with acetic acid are combined to generate formaldehyde in situ. The aldol condensation then follows. In one embodiment, a methanol-formaldehyde solution is reacted with acetic acid to form the crude acrylate product.

The alkanoic acid, or an ester of the alkanoic acid, may be of the formula R′—CH₂—COOR, where R and R′ are each, independently, hydrogen or a saturated or unsaturated alkyl or aryl group. As an example, R and R′ may be a lower alkyl group containing for example 1-4 carbon atoms. In one embodiment, an alkanoic acid anhydride may be used as the source of the alkanoic acid. In one embodiment, the reaction is conducted in the presence of an alcohol, preferably the alcohol that corresponds to the desired ester, e.g., methanol. In addition to reactions used in the production of acrylic acid, the inventive catalyst, in other embodiments, may be employed to catalyze other reactions.

The alkanoic acid, e.g., acetic acid, may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. As petroleum and natural gas prices fluctuate, becoming either more or less expensive, methods for producing acetic acid and intermediates such as methanol and carbon monoxide from alternate carbon sources have drawn increasing interest. In particular, when petroleum is relatively expensive compared to natural gas, it may become advantageous to produce acetic acid from synthesis gas (“syngas”) that is derived from any available carbon source. U.S. Pat. No. 6,232,352, which is hereby incorporated by reference, for example, teaches a method of retrofitting a methanol plant for the manufacture of acetic acid. By retrofitting a methanol plant, the large capital costs associated with carbon monoxide generation for a new acetic acid plant are significantly reduced or largely eliminated. All or part of the syn gas is diverted from the methanol synthesis loop and supplied to a separator unit to recover carbon monoxide and hydrogen, which are then used to produce acetic acid.

In some embodiments, at least some of the raw materials for the above-described aldol condensation process may be derived partially or entirely from syngas. For example, the acetic acid may be formed from methanol and carbon monoxide, both of which may be derived from syngas. For example, the methanol may be formed by steam reforming syngas, and the carbon monoxide may be separated from syngas. In other embodiments, the methanol may be formed in a carbon monoxide unit, e.g., as described in EP2076480; EP1923380; EP2072490; EP1914219; EP1904426; EP2072487; EO2072492; EP2072486; EP2060553; EP1741692; EP1907344; EP2060555; EP2186787; EP2072488; and U.S. Pat. No. 7,842,844, which are hereby incorporated by reference. Of course, this listing of methanol sources is merely exemplary and is not meant to be limiting. In addition, the above-identified methanol sources, inter alia, may be used to form the formaldehyde, e.g., in situ, which, in turn may be reacted with the acetic acid to form the acrylic acid. The syngas, in turn, may be derived from variety of carbon sources. The carbon source, for example, may be selected from the group consisting of natural gas, oil, petroleum, coal, biomass, and combinations thereof.

Methanol carbonylation processes suitable for production of acetic acid are described in U.S. Pat. Nos. 7,208,624, 7,115,772, 7,005,541, 6,657,078, 6,627,770, 6,143,930, 5,599,976, 5,144,068, 5,026,908, 5,001,259, and 4,994,608, all of which are hereby incorporated by reference.

U.S. Pat. No. RE 35,377, which is hereby incorporated by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syn gas. The syn gas is converted to methanol which may be carbonylated to acetic acid. U.S. Pat. No. 5,821,111, which discloses a process for converting waste biomass through gasification into syn gas, as well as U.S. Pat. No. 6,685,754 are hereby incorporated by reference.

In one optional embodiment, the acetic acid that is utilized in the condensation reaction comprises acetic acid and may also comprise other carboxylic acids, e.g., propionic acid, esters, and anhydrides, as well as acetaldehyde and acetone. In one embodiment, the acetic acid fed to the condensation reaction comprises propionic acid. For example, the acetic acid fed to the reaction may comprise from 0.001 wt. % to 15 wt. % propionic acid, e.g., from 0.001 wt. % to 0.11 wt. %, from 0.125 wt. % to 12.5 wt. %, from 1.25 wt. % to 11.25 wt. %, or from 3.75 wt. % to 8.75 wt. %. Thus, the acetic acid feed stream may be a cruder acetic acid feed stream, e.g., a less-refined acetic acid feed stream.

As used herein, “alkylenating agent” means an aldehyde or precursor to an aldehyde suitable for reacting with the alkanoic acid, e.g., acetic acid, to form an unsaturated acid, e.g., acrylic acid, or an alkyl acrylate. In preferred embodiments, the alkylenating agent comprises a methylenating agent such as formaldehyde, which preferably is capable of adding a methylene group (═CH₂) to the organic acid. Other alkylenating agents may include, for example, acetaldehyde, propanal, butanal, aryl aldehydes, benzyl aldehydes, alcohols, and combinations thereof. This listing is not exclusive and is not meant to limit the scope of the invention. In one embodiment, an alcohol may serve as a source of the alkylenating agent. For example, the alcohol may be reacted in situ to form the alkylenating agent, e.g., the aldehyde.

The alkylenating agent, e.g., formaldehyde, may be derived from any suitable source. Exemplary sources may include, for example, aqueous formaldehyde solutions, anhydrous formaldehyde derived from a formaldehyde drying procedure, trioxane, diether of methylene glycol, and paraformaldehyde. In a preferred embodiment, the formaldehyde is produced via a methanol oxidation process, which reacts methanol and oxygen to yield the formaldehyde.

In other embodiments, the alkylenating agent is a compound that is a source of formaldehyde. Where forms of formaldehyde that are not as freely or weakly complexed are used, the formaldehyde will form in situ in the condensation reactor or in a separate reactor prior to the condensation reactor. Thus for example, trioxane may be decomposed over an inert material or in an empty tube at temperatures over 350° C. or over an acid catalyst at over 100° C. to form the formaldehyde.

In one embodiment, the alkylenating agent corresponds to Formula I.

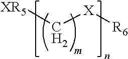

In this formula, R₅ and R₆ may be independently selected from C₁-C₁₂ hydrocarbons, preferably, C₁-C₁₂ alkyl, alkenyl or aryl, or hydrogen. Preferably, R₅ and R₆ are independently C₁-C₆ alkyl or hydrogen, with methyl and/or hydrogen being most preferred. X may be either oxygen or sulfur, preferably oxygen; and n is an integer from 1 to 10, preferably 1 to 3. In some embodiments, m is 1 or 2, preferably 1.

In one embodiment, the compound of formula I may be the product of an equilibrium reaction between formaldehyde and methanol in the presence of water. In such a case, the compound of formula I may be a suitable formaldehyde source. In one embodiment, the formaldehyde source includes any equilibrium composition. Examples of formaldehyde sources include but are not restricted to methylal (1,1 dimethoxymethane); polyoxymethylenes —(CH₂—O)ᵢ— wherein i is from 1 to 100; formalin; and other equilibrium compositions such as a mixture of formaldehyde, methanol, and methyl propionate. In one embodiment, the source of formaldehyde is selected from the group consisting of 1, 1 dimethoxymethane; higher formals of formaldehyde and methanol; and CH₃—O—(CH₂—O)ᵢ—CH₃ where i is 2.

The alkylenating agent may be used with or without an organic or inorganic solvent.

The term “formalin,” refers to a mixture of formaldehyde, methanol, and water. In one embodiment, formalin comprises from 25 wt. % to 65% formaldehyde; from 0.01 wt. % to 25 wt. % methanol; and from 25 wt. % to 70 wt. % water. In cases where a mixture of formaldehyde, methanol, and methyl propionate is used, the mixture comprises less than 10 wt. % water, e.g., less than 5 wt. % or less than 1 wt. %.

In some embodiments, the condensation reaction may achieve favorable conversion of acetic acid and favorable selectivity and productivity to acrylates. For purposes of the present invention, the term “conversion” refers to the amount of acetic acid in the feed that is converted to a compound other than acetic acid. Conversion is expressed as a percentage based on acetic acid in the feed. The conversion of acetic acid may be at least 10%, e.g., at least 20%, at least 40%, or at least 50%.

Selectivity, as it refers to the formation of acrylate product, is expressed as the ratio of the amount of carbon in the desired product(s) and the amount of carbon in the total products. This ratio may be multiplied by 100 to arrive at the selectivity. Preferably, the catalyst selectivity to acrylate products, e.g., acrylic acid and methyl acrylate, is at least 40 mol %, e.g., at least 50 mol %, at least 60 mol %, or at least 70 mol %. In some embodiments, the selectivity to acrylic acid is at least 30 mol %, e.g., at least 40 mol %, or at least 50 mol %; and/or the selectivity to methyl acrylate is at least 10 mol %, e.g., at least 15 mol %, or at least 20 mol %.

The terms “productivity” or “space time yield” as used herein, refers to the grams of a specified product, e.g., acrylate products, formed per hour during the condensation based on the liters of catalyst used. A productivity of at least 20 grams of acrylate product per liter catalyst per hour, e.g., at least 40 grams of acrylates per liter catalyst per hour or at least 100 grams of acrylates per liter catalyst per hour, is preferred. In taints of ranges, the productivity preferably is from 20 to 500 grams of acrylates per liter catalyst per hour, e.g., from 20 to 200 grams of acrylates per liter catalyst per hour or from 40 to 140 grams of acrylates per liter catalyst per hour.

In one embodiment, the inventive process yields at least 1,800 kg/hr of finished acrylic acid, e.g., at least 3,500 kg/hr, at least 18,000 kg/hr, or at least 37,000 kg/hr.

Preferred embodiments of the inventive process demonstrate a low selectivity to undesirable products, such as carbon monoxide and carbon dioxide. The selectivity to these undesirable products preferably is less than 29%, e.g., less than 25% or less than 15%. More preferably, these undesirable products are not detectable. Formation of alkanes, e.g., ethane, may be low, and ideally less than 2%, less than 1%, or less than 0.5% of the acetic acid passed over the catalyst is converted to alkanes, which have little value other than as fuel.

The alkanoic acid or ester thereof and alkylenating agent may be fed independently or after prior mixing to a reactor containing the catalyst. The reactor may be any suitable reactor or combination of reactors. Preferably, the reactor comprises a fixed bed reactor or a series of fixed bed reactors. In one embodiment, the reactor is a packed bed reactor or a series of packed bed reactors. In one embodiment, the reactor is a fixed bed reactor. Of course, other reactors such as a continuous stirred tank reactor or a fluidized bed reactor, may be employed.

In some embodiments, the alkanoic acid, e.g., acetic acid, and the alkylenating agent, e.g., formaldehyde, are fed to the reactor at a molar ratio of at least 0.10:1, e.g., at least 0.75:1 or at least 1:1. In terms of ranges the molar ratio of alkanoic acid to alkylenating agent may range from 0.10:1 to 10:1 or from 0.75:1 to 5:1. In some embodiments, the reaction of the alkanoic acid and the alkylenating agent is conducted with a stoichiometric excess of alkanoic acid. In these instances, acrylate selectivity may be improved. As an example the acrylate selectivity may be at least 10% higher than a selectivity achieved when the reaction is conducted with an excess of alkylenating agent, e.g., at least 20% higher or at least 30% higher. In other embodiments, the reaction of the alkanoic acid and the alkylenating agent is conducted with a stoichiometric excess of alkylenating agent.

The condensation reaction may be conducted at a temperature of at least 250° C., e.g., at least 300° C., or at least 350° C. In terms of ranges, the reaction temperature may range from 200° C. to 500° C., e.g., from 250° C. to 400° C., or from 250° C. to 350° C. The acetic acid conversion, in some embodiments, may vary depending upon the reaction temperature. Residence time in the reactor may range from 1 second to 200 seconds, e.g., from 1 second to 100 seconds. Reaction pressure is not particularly limited, and the reaction is typically performed near atmospheric pressure. In one embodiment, the reaction may be conducted at a pressure ranging from 0 kPa to 4100 kPa, e.g., from 3 kPa to 345 kPa, or from 6 to 103 kPa. Without wishing to be bound by any theory, it is believed that, with chemical kinetics of the reaction chemistry as a driver, reaction efficiency may be improved with lower reactant concentrations (in mol/m³). Decreased reaction pressure, with corresponding decreased reactant concentrations (i.e., partial pressures), results in greater product yields. In one embodiment, addition of one or more diluents, e.g., nitrogen and/or carbon dioxide, to the reaction mixture can further reduce reactant concentrations (i.e., partial pressures) in the reaction mixture. While lower operating pressures and/or inclusion of diluent(s) in the reaction mixture will increase product yield, overall production per unit volume of catalyst will be decreased, due to the lower operating pressure and/or dilution of the reaction mixture. Again, without wishing to be bound by any theory, in one embodiment, it is believed that the produced acrylate product(s), for example, acrylic acid, may act as an inhibitor for the catalyst of the presently disclosed process.

In one embodiment, the reaction is conducted at a gas hourly space velocity (“GHSV”) greater than 600 hr⁻¹, e.g., greater than 1000 hr⁻¹ or greater than 2000 hr⁻¹. In one embodiment, the GHSV ranges from 600 hr⁻¹ to 10000 hr⁻¹, e.g., from 1000 hr⁻¹ to 8000 hr⁻¹ or from 1500 hr⁻¹ to 7500 hr⁻¹. As one particular example, when GHSV is at least 2000 hr⁻¹, the acrylate product STY may be at least 150 g/hr/liter.

Water may be present in the reactor in amounts up to 60 wt. %, by weight of the reaction mixture, e.g., up to 50 wt. % or up to 40 wt. %. Water, however, is preferably reduced due to its negative effect on process rates and separation costs.

In one embodiment, an inert or reactive gas, e.g., a diluent, is supplied to the reactant stream. Examples of inert gases include, but are not limited to, nitrogen, helium, argon, and methane. Examples of reactive gases or vapors include, but are not limited to, oxygen, carbon oxides, sulfur oxides, and alkyl halides. When reactive gases such as oxygen are added to the reactor, these gases, in some embodiments, may be added in stages throughout the catalyst bed at desired levels as well as feeding with the other feed components at the beginning of the reactors. The addition of these additional components may improve reaction efficiencies.

In one embodiment, the unreacted components such as the alkanoic acid and formaldehyde as well as the inert or reactive gases that remain are recycled to the reactor after sufficient separation from the desired product.

When the desired product is an unsaturated ester made by reacting an ester of an alkanoic acid ester with formaldehyde, the alcohol corresponding to the ester may also be fed to the reactor either with or separately to the other components. For example, when methyl acrylate is desired, methanol may be fed to the reactor. The alcohol, amongst other effects, reduces the quantity of acids leaving the reactor. It is not necessary that the alcohol is added at the beginning of the reactor and it may for instance be added in the middle or near the back, in order to effect the conversion of acids such as propionic acid, methacrylic acid to their respective esters without depressing catalyst activity. In one embodiment, the alcohol may be added downstream of the reactor.

### Catalyst Composition

The catalyst may be any suitable catalyst composition. As one example, condensation catalyst consisting of mixed oxides of vanadium and phosphorus have been investigated and described in M. Ai, J. Catal., 107,201 (1987); M. Ai, J. Catal., 124, 293 (1990); M. Ai, Appl. Catal., 36, 221 (1988); and M. Ai, Shokubai, 29, 522 (1987). Other examples include binary vanadium-titanium phosphates, vanadium-silica-phosphates, and alkali metal-promoted silicas, e.g., cesium- or potassium-promoted silicas.

In a preferred embodiment, the inventive process employs a catalyst composition comprising vanadium, titanium, and optionally at least one oxide additive. The oxide additive(s), if present, are preferably present in the active phase of the catalyst. In one embodiment, the oxide additive(s) are selected from the group consisting of silica, alumina, zirconia, and mixtures thereof or any other metal oxide other than metal oxides of titanium or vanadium. Preferably, the molar ratio of oxide additive to titanium in the active phase of the catalyst composition is greater than 0.05:1, e.g., greater than 0.1:1, greater than 0.5:1, or greater than 1:1. In terms of ranges, the molar ratio of oxide additive to titanium in the inventive catalyst may range from 0.05:1 to 20:1, e.g., from 0.1:1 to 10:1, or from 1:1 to 10:1. In these embodiments, the catalyst comprises titanium, vanadium, and one or more oxide additives and have relatively high molar ratios of oxide additive to titanium.

In another embodiment, the inventive process employs a catalyst comprising vanadium, titanium, bismuth, tungsten, or mixtures thereof. In some embodiments, the catalyst comprises bismuth. In other embodiments, the catalyst comprises tungsten. Exemplary catalyst compositions include vanadium/titanium/bismuth, vanadium/titanium/tungsten, bismuth/tungsten, vanadium/bismuth, vanadium/tungsten, and vanadium/bismuth/tungsten.

In other embodiments, the catalyst may further comprise other compounds or elements (metals and/or non-metals). For example, the catalyst may further comprise phosphorus and/or oxygen. In these cases, the catalyst may comprise from 15 wt. % to 45 wt. % phosphorus, e.g., from 20 wt. % to 35 wt. % or from 23 wt. % to 27 wt. %; and/or from 30 wt. % to 75 wt. % oxygen, e.g., from 35 wt. % to 65 wt. % or from 48 wt. % to 51 wt. %.

In some embodiments, the catalyst further comprises additional metals and/or oxide additives. These additional metals and/or oxide additives may function as promoters. If present, the additional metals and/or oxide additives may be selected from the group consisting of copper, molybdenum, tungsten, nickel, niobium, and combinations thereof. Other exemplary promoters that may be included in the catalyst of the invention include lithium, sodium, magnesium, aluminum, chromium, manganese, iron, cobalt, calcium, yttrium, ruthenium, silver, tin, barium, lanthanum, the rare earth metals, hafnium, tantalum, rhenium, thorium, bismuth, antimony, germanium, zirconium, uranium, cesium, zinc, and silicon and mixtures thereof. Other modifiers include boron, gallium, arsenic, sulfur, halides, Lewis acids such as BF₃, ZnBr₂, and SnCl₄. Exemplary processes for incorporating promoters into catalyst are described in U.S. Pat. No. 5,364,824, the entirety of which is incorporated herein by reference.

If the catalyst comprises additional metal(s) and/or metal oxides(s), the catalyst optionally may comprise additional metals and/or metal oxides in an amount from 0.001 wt. % to 30 wt. %, e.g., from 0.01 wt. % to 5 wt. % or from 0.1 wt. % to 5 wt. %. If present, the promoters may enable the catalyst to have a weight/weight space time yield of at least 25 grams of acrylic acid/gram catalyst-h, e.g., least 50 grams of acrylic acid/gram catalyst-h, or at least 100 grams of acrylic acid/gram catalyst-h.

In some embodiments, the catalyst is unsupported. In these cases, the catalyst may comprise a homogeneous mixture or a heterogeneous mixture as described above. In one embodiment, the homogeneous mixture is the product of an intimate mixture of vanadium and titanium oxides, hydroxides, and phosphates resulting from preparative methods such as controlled hydrolysis of metal alkoxides or metal complexes. In other embodiments, the heterogeneous mixture is the product of a physical mixture of the vanadium and titanium phosphates. These mixtures may include formulations prepared from phosphorylating a physical mixture of preformed hydrous metal oxides. In other cases, the mixture(s) may include a mixture of preformed vanadium pyrophosphate and titanium pyrophosphate powders.

In another embodiment, the catalyst is a supported catalyst comprising a catalyst support in addition to the vanadium, titanium, oxide additive, and optionally phosphorous and oxygen, in the amounts indicated above (wherein the molar ranges indicated are without regard to the moles of catalyst support, including any vanadium, titanium, oxide additive, phosphorous or oxygen contained in the catalyst support). The total weight of the support (or modified support), based on the total weight of the catalyst, preferably is from 75 wt. % to 99.9 wt. %, e.g., from 78 wt. % to 97 wt. % or from 80 wt. % to 95 wt. %. The support may vary widely. In one embodiment, the support material is selected from the group consisting of silica, alumina, zirconia, titania, aluminosilicates, zeolitic materials, mixed metal oxides (including but not limited to binary oxides such as SiO₂—Al₂O₃, SiO₂—TiO₂, SiO₂—ZnO, SiO₂—MgO, SiO₂—ZrO₂, Al₂O₃—MgO, Al₂O₃—TiO₂, Al₂O₃—ZnO, TiO₂—MgO, TiO₂—ZrO₂, TiO₂—ZnO, TiO₂—SnO₂) and mixtures thereof, with silica being one preferred support. In embodiments where the catalyst comprises a titania support, the titania support may comprise a major or minor amount of rutile and/or anatase titanium dioxide. Other suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, silicon carbide, sheet silicates or clay minerals such as montmorillonite, beidellite, saponite, pillared clays, other microporous and mesoporous materials, and mixtures thereof. Other supports may include, but are not limited to, iron oxide, magnesia, steatite, magnesium oxide, carbon, graphite, high surface area graphitized carbon, activated carbons, and mixtures thereof. These listings of supports are merely exemplary and are not meant to limit the scope of the present invention.

In some embodiments, a zeolitic support is employed. For example, the zeolitic support may be selected from the group consisting of montmorillonite, NH₄ ferrierite, H-mordenite-PVOx, vermiculite-1, H-ZSM5, NaY, H-SDUSY, Y zeolite with high SAR, activated bentonite, H-USY, MONT-2, HY, mordenite SAR 20, SAPO-34, Aluminosilicate (X), VUSY, Aluminosilicate (CaX), Re-Y, and mixtures thereof. H-SDUSY, VUSY, and H-USY are modified Y zeolites belonging to the faujasite family. In one embodiment, the support is a zeolite that does not contain any metal oxide modifier(s). In some embodiments, the catalyst composition comprises a zeolitic support and the active phase comprises a metal selected from the group consisting of vanadium, aluminum, nickel, molybdenum, cobalt, iron, tungsten, zinc, copper, titanium cesium bismuth, sodium, calcium, chromium, cadmium, zirconium, and mixtures thereof. In some of these embodiments, the active phase may also comprise hydrogen, oxygen, and/or phosphorus.

In other embodiments, in addition to the active phase and a support, the inventive catalyst may further comprise a support modifier. A modified support, in one embodiment, relates to a support that includes a support material and a support modifier, which, for example, may adjust the chemical or physical properties of the support material such as the acidity or basicity of the support material. In embodiments that use a modified support, the support modifier is present in an amount from 0.1 wt. % to 50 wt. %, e.g., from 0.2 wt. % to 25 wt. %, from 0.5 wt. % to 15 wt. %, or from 1 wt. % to 8 wt. %, based on the total weight of the catalyst composition.

In one embodiment, the support modifier is an acidic support modifier. In some embodiments, the catalyst support is modified with an acidic support modifier. The support modifier similarly may be an acidic modifier that has a low volatility or little volatility. The acidic modifiers may be selected from the group consisting of oxides of Group IVB metals, oxides of Group VB metals, oxides of Group VIB metals, iron oxides, aluminum oxides, and mixtures thereof. In one embodiment, the acidic modifier may be selected from the group consisting of WO₃, MoO₃, Fe₂O₃, Cr₂O₃, V₂O₅, MnO₂, CuO, Co₂O₃, Bi₂O₃, TiO₂, ZrO₂, Nb₂O₅, Ta₂O₅, Al₂O₃, B₂O₃, P₂O₅, and Sb₂O₃.

In another embodiment, the support modifier is a basic support modifier. The presence of chemical species such as alkali and alkaline earth metals, are normally considered basic and may conventionally be considered detrimental to catalyst performance. The presence of these species, however, surprisingly and unexpectedly, may be beneficial to the catalyst performance. In some embodiments, these species may act as catalyst promoters or a necessary part of the acidic catalyst structure such in layered or sheet silicates such as montmorillonite. Without being bound by theory, it is postulated that these cations create a strong dipole with species that create acidity.

Additional modifiers that may be included in the catalyst include, for example, boron, aluminum, magnesium, zirconium, and hafnium.

As will be appreciated by those of ordinary skill in the art, the support materials, if included in the catalyst of the present invention, preferably are selected such that the catalyst system is suitably active, selective and robust under the process conditions employed for the formation of the desired product, e.g., acrylic acid or alkyl acrylate. Also, the active metals and/or pyrophosphates that are included in the catalyst of the invention may be dispersed throughout the support, coated on the outer surface of the support (egg shell) or decorated on the surface of the support. In some embodiments, in the case of macro- and meso-porous materials, the active sites may be anchored or applied to the surfaces of the pores that are distributed throughout the particle and hence are surface sites available to the reactants but are distributed throughout the support particle.

The inventive catalyst may further comprise other additives, examples of which may include: molding assistants for enhancing moldability; reinforcements for enhancing the strength of the catalyst; pore-forming or pore modification agents for formation of appropriate pores in the catalyst, and binders. Examples of these other additives include stearic acid, graphite, starch, cellulose, silica, alumina, glass fibers, silicon carbide, and silicon nitride. Preferably, these additives do not have detrimental effects on the catalytic performances, e.g., conversion and/or activity. These various additives may be added in such an amount that the physical strength of the catalyst does not readily deteriorate to such an extent that it becomes impossible to use the catalyst practically as an industrial catalyst.

### Separation

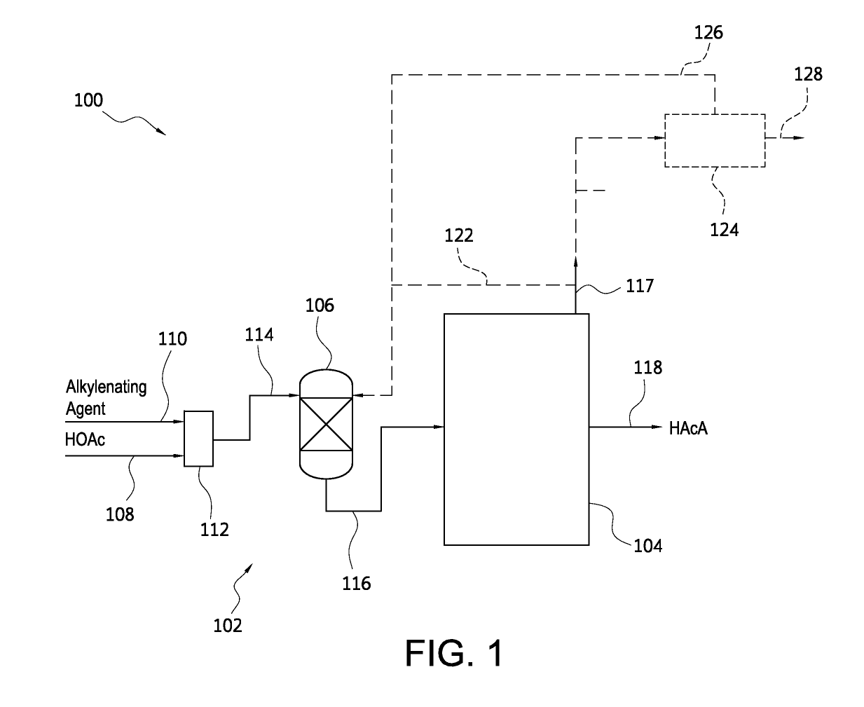

The unique crude acrylate stream of the present invention may be separated in a separation zone to form a final product, e.g., a purified acrylate product. FIG. 1 is a flow diagram depicting the formation of the crude acrylate stream and the separation thereof to obtain a purified acrylate product 118. Acrylate product system 100 comprises reaction zone 102 and separation zone 104. Reaction zone 102 comprises reactor 106, alkanoic acid feed, e.g., acetic acid feed, 108, alkylenating agent feed, e.g., formaldehyde feed 110, and vaporizer 112.

Acetic acid and formaldehyde are fed to vaporizer 112 via lines 108 and 110, respectively, to create a vapor feed stream, which exits vaporizer 112 via line 114 and is directed to reactor 106. In one embodiment, lines 108 and 110 may be combined and jointly fed to the vaporizer 112. The temperature of the vapor feed stream in line 114 is preferably from 200° C. to 600° C., e.g., from 250° C. to 500° C. or from 340° C. to 425° C. Alternatively, a vaporizer may not be employed and the reactants may be fed directly to reactor 106.

Any feed that is not vaporized may be removed from vaporizer 112 and may be recycled or discarded. In addition, although line 114 is shown as being directed to the upper half of reactor 106, line 114 may be directed to the middle or bottom of first reactor 106. Further modifications and additional components to reaction zone 102 and separation zone 104 are described below.

Reactor 106 contains the catalyst that is used in the reaction to form crude acrylate product, which is withdrawn, preferably continuously, from reactor 106 via line 116. Although FIG. 1 shows the crude acrylate product being withdrawn from the bottom of reactor 106, the crude acrylate product may be withdrawn from any portion of reactor 106. Exemplary composition ranges for the crude acrylate product are shown in Table 1 above.

In one embodiment, one or more guard beds (not shown) may be used upstream of the reactor to protect the catalyst from poisons or undesirable impurities contained in the feed or return/purge streams. Such guard beds may be employed in the vapor or liquid acrylate streams. Suitable guard bed materials may include, for example, carbon, silica, alumina, ceramic, or resins. In one aspect, the guard bed media is functionalized, e.g., silver functionalized, to trap particular species such as sulfur or halogens.

Crude acrylate product in line 116 is fed to separation zone 104. Separation zone 104 may comprise one or more separation units, e.g., two or more or three or more. As discussed above, some of the components of the crude acrylate product may react, under certain conditions, to form an intermediate acrylate product comprising, inter alia, by-products, e.g., Michael addition by-products. This reaction, in some embodiments, may occur in separation zone 104. Separation zone 104 separates the intermediate acrylate product, thus formed, to yield a purified acrylate product, which exits via line 118, and by-product stream, which exits via line 117.

By-product stream 117 may be utilized elsewhere in the system. Optionally, at least a portion of by-product stream 117 may be directed to reactor 106 via optional line 122 for further reaction, e.g., cracking or decomposition.

In one embodiment, at least a portion of by-product stream 117, e.g., the Michael addition product, may be reacted, e.g., cracked or decomposed, in optional reactor 124. Reactor 124 may yield the resultant cracked products, e.g., acetic acid and acrylic acid. These cracked products may optionally be directed to reactor 106 via line 126 or may be directed to other separation units of separation zone 104 via line 128.

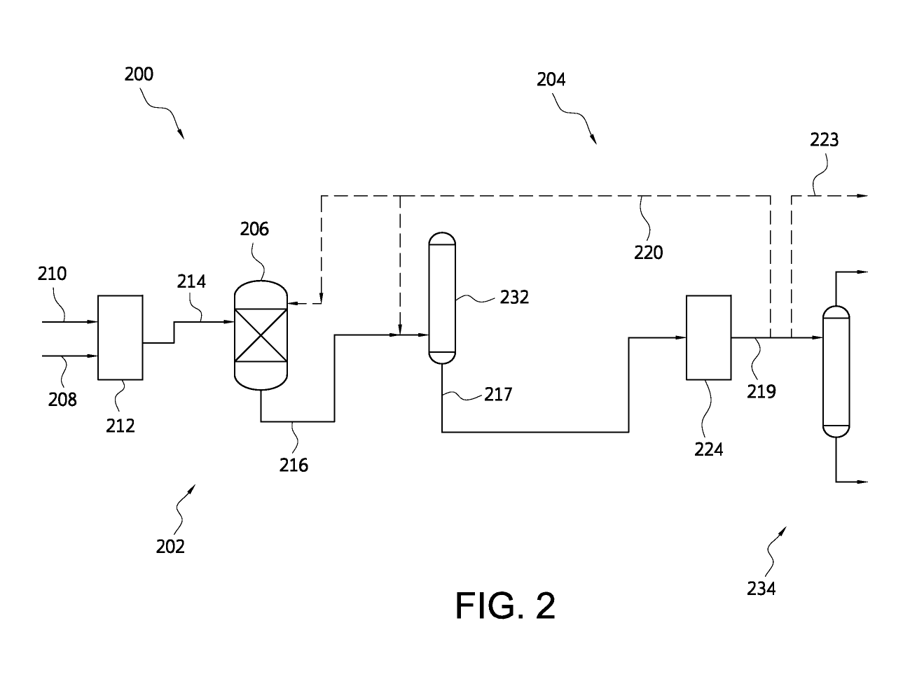

FIG. 2 shows an overview of an alternative reaction/separation scheme in accordance with the present invention. Acrylate product system 200 comprises reaction zone 202 and separation zone 204. Reaction zone 202 comprises reactor 206, alkanoic acid feed, e.g., acetic acid feed, 208, alkylenating agent feed, e.g., formaldehyde feed, 210, vaporizer 212, and line 214. Reaction zone 202 and the components thereof function in a manner similar to reaction zone 102 of FIG. 1. Reactor 206 contains the catalyst that is used in the reaction to form crude acrylate product, which is withdrawn, preferably continuously, from reactor 206 via line 216.

Reaction zone 202 yields a crude acrylate product, which exits reaction zone 202 via line 216 and is directed to separation zone 204. The components of the crude acrylate product are discussed above.

In one example, separation zone 204 contains multiple columns, as shown in FIG. 2. Separation zone 204 comprises alkylenating agent split unit 232, acrylate product split unit 234. Separation zone may, in some embodiments, further comprise a drying unit and/or a methanol removal unit (units not shown in FIG. 2). As noted above, some components of the crude acrylate product may, under some conditions, react to form by-products. In one embodiment, acrylic acid and acetic acid may undergo a Michael addition reaction to form Michael addition by-products. In one embodiment, such a reaction occurs in alkylenating agent split unit 232. The by-products may combine with the components of the crude acrylate product to form an intermediate acrylate product, which may be then separated in alkylenating agent split unit 232 to yield a by-product stream, which exits alkylenating agent split unit 232 via line 217. The composition of by-product stream 217 may be as discussed above. Optionally, at least a portion of by-product stream 217 may be directed to reactor 206 for further reaction, e.g., cracking or decomposition.

In one embodiment, at least a portion of by-product stream 217, e.g., the Michael addition product, may be reacted, e.g., cracked or decomposed, in reactor 224. Reactor 224 may yield the resultant (recovered) cracked products, e.g., acetic acid and acrylic acid. These (recovered) cracked products may optionally be directed via line 220 to reactor 206 or may be directed to other separation units of separation zone 104. For example, at least a portion of the cracked products may be directed via line 219 to a second column, e.g., acrylate product split unit 234. In one embodiment, at least a portion of the cracked products may optionally be directed back to alkylenating agent split unit 232 via line 220.

In other embodiments, at least a portion of the cracked products may be directed via line 223 to another component of the separation zone (other components not shown in FIG. 2). Of course, the present invention contemplates the use of a wide range of separation schemes in addition to those disclosed in FIGS. 1 and 2.

Alkylenating agent split unit 232 may comprise any suitable separation device or combination of separation devices. For example, alkylenating agent split unit 232 may comprise a column, e.g., a standard distillation column, an extractive distillation column and/or an azeotropic distillation column. In other embodiments, alkylenating agent split unit 232 comprises a precipitation unit, e.g., a crystallizer and/or a chiller. Preferably, alkylenating agent split unit 232 comprises a single distillation column.

In another embodiment, the alkylenating agent split is performed by contacting the crude acrylate product with a solvent that is immiscible with water. For example, alkylenating agent split unit 232 may comprise at least one liquid-liquid extraction column. In another embodiment, the alkylenating agent split is performed via azeotropic distillation, which employs an azeotropic agent. In these cases, the azeotropic agent may be selected from the group consisting of methyl isobutylketene, o-xylene, toluene, benzene, n-hexane, cyclohexane, p-xylene, and mixtures thereof. This listing is not exclusive and is not meant to limit the scope of the invention. In another embodiment, the alkylenating agent split is performed via a combination of distillations, e.g., standard distillation, and crystallization. Of course, other suitable separation devices may be employed either alone or in combination with the devices mentioned herein.

In one embodiment, polymerization inhibitors and/or anti-foam agents may be employed in the separation zone, e.g., in the units of the separation zone. The inhibitors may be used to reduce the potential for fouling caused by polymerization of acrylates. The anti-foam agents may be used to reduce potential for foaming in the various streams of the separation zone. The polymerization inhibitors and/or the anti-foam agents may be used at one or more locations in the separation zone.

In cases where any of alkylenating agent split unit 232 comprises at least one column, the column(s) may be operated at suitable temperatures and pressures, e.g., the Michael addition reaction temperatures mentioned above. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 300 kPa, e.g., from 10 kPa to 100 kPa or from 40 kPa to 80 kPa. In preferred embodiments, the pressure at which the column(s) are operated is kept at a low level e.g., less than 100 kPa, less than 80 kPa, or less than 60 kPa. In terms of lower limits, the column(s) may be operated at a pressures of at least 1 kPa, e.g., at least 20 kPa or at least 40 kPa. Without being bound by theory, it is believed that alkylenating agents, e.g., formaldehyde, may not be sufficiently volatile at lower pressures. Thus, maintenance of the column pressures at these levels surprisingly and unexpectedly provides for efficient separation operations. In addition, it has surprisingly and unexpectedly been found that by maintaining a low pressure in the columns of alkylenating agent split unit 232 may inhibit and/or eliminate polymerization of the acrylate products, e.g., acrylic acid, which may contribute to fouling of the column(s).

In one embodiment, the alkylenating agent split is achieved via one or more liquid-liquid extraction units. Preferably, the one or more liquid-liquid extraction units employ one or more extraction agents. Multiple liquid-liquid extraction units may be employed to achieve the alkylenating agent split. Any suitable liquid-liquid extraction devices used for multiple equilibrium stage separations may be used. Also, other separation devices, e.g., traditional columns, may be employed in conjunction with the liquid-liquid extraction unit(s).

In one embodiment (not shown), the crude acrylate product is fed to a liquid-liquid extraction column where the crude acrylate product is contacted with an extraction agent, e.g., an organic solvent. The liquid-liquid extraction column extracts the acids, e.g., acrylic acid and acetic acid, from the crude acrylate product. An aqueous phase comprising water, alkylenating agent, and some acetic acid exits the liquid-liquid extraction unit. Small amounts of acrylic acid may also be present in the aqueous stream. The aqueous phase may be further treated and/or recycled. An organic phase comprising acrylic acid, acetic acid, and the extraction agent also exits the liquid-liquid extraction unit. The organic phase may also comprise water and formaldehyde. The acrylic acid may be separated from the organic phase and collected as product. The acetic acid may be separated then recycled and/or used elsewhere. The solvent may be recovered and recycled to the liquid-liquid extraction unit.

Acrylate product split unit 234 may comprise any suitable separation device or combination of separation devices. For example, acrylate product split unit 234 may comprise at least one column, e.g., a standard distillation column, an extractive distillation column and/or an azeotropic distillation column. In other embodiments, acrylate product split unit 234 comprises a precipitation unit, e.g., a crystallizer and/or a chiller. In another embodiment, acrylate product split unit 234 comprises a liquid-liquid extraction unit. Of course, other suitable separation devices may be employed either alone or in combination with the devices mentioned herein.

In cases where the acrylate product split unit comprises at least one column, the column(s) may be operated at suitable temperatures and pressures, e.g., the Michael addition reaction temperatures mentioned above. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 300 kPa, e.g., from 10 kPa to 100 kPa or from 40 kPa to 80 kPa. In preferred embodiments, the pressure at which the column(s) are operated is kept at a low level e.g., less than 50 kPa, less than 27 kPa, or less than 20 kPa. In terms of lower limits, the column(s) may be operated at a pressures of at least 1 kPa, e.g., at least 3 kPa or at least 5 kPa. Without being bound by theory, it has surprisingly and unexpectedly been found that be maintaining a low pressure in the columns of acrylate product split unit 234 may inhibit and/or eliminate polymerization of the acrylate products, e.g., acrylic acid, which may contribute to fouling of the column(s).

It has also been found that, surprisingly and unexpectedly, maintaining the temperature of acrylic acid-containing streams fed to acrylate product split unit 234 at temperatures below 140° C., e.g., below 130° C. or below 115° C., may inhibit and/or eliminate polymerization of acrylate products. In one embodiment, to maintain the liquid temperature at these temperatures, the pressure of the column(s) is maintained at or below the pressures mentioned above. In these cases, due to the lower pressures, the number of theoretical column trays is kept at a low level, e.g., less than 10, less than 8, less than 7, or less than 5. As such, it has surprisingly and unexpectedly been found that multiple columns having fewer trays inhibit and/or eliminate acrylate product polymerization. In contrast, a column having a higher amount of trays, e.g., more than 10 trays or more than 15 trays, would suffer from fouling due to the polymerization of the acrylate products. Thus, in a preferred embodiment, the acrylic acid split is performed in at least two, e.g., at least three, columns, each of which have less than 10 trays, e.g. less than 7 trays. These columns each may operate at the lower pressures discussed above.

With respect to the alkylenating agent split, as noted above, the presence of alkylenating agent in the crude acrylate stream adds unpredictability and problems to separation schemes. Without being bound by theory, it is believed that formaldehyde reacts in many side reactions with water to form by-products. The following side reactions are exemplary.

CH₂O+H₂O→HOCH₂OH

HO(CH₂O)ᵢ₋₁H+HOCH₂OH→HO(CH₂O)ᵢH+H₂O for i>1

Without being bound by theory, it is believed that, in some embodiments, as a result of these reactions, the alkylenating agent, e.g., formaldehyde, acts as a “light” component at higher temperatures and as a “heavy” component at lower temperatures. The reaction(s) are exothermic. Accordingly, the equilibrium constant increases as temperature decreases and decreases as temperature increases. At lower temperatures, the larger equilibrium constant favors methylene glycol and oligomer production and formaldehyde becomes limited, and, as such, behaves as a heavy component. At higher temperatures, the smaller equilibrium constant favors formaldehyde production and methylene glycol becomes limited. As such, formaldehyde behaves as a light component. In view of these difficulties, as well as others, the separation of streams that comprise water and formaldehyde cannot be expected to behave as a typical two-component system. These features contribute to the unpredictability and difficulty of the separation of the unique diluted crude acrylate stream of the present invention.

The present invention, surprisingly and unexpectedly, achieves effective separation of alkylenating agent(s) from the inventive crude acrylate stream to yield a purified product comprising acrylate product and very low amounts of other impurities.

In one embodiment, the crude acrylate stream is optionally treated, e.g. separated, prior to the separation of alkylenating agent therefrom. In such cases, the treatment(s) occur before the alkylenating agent split is performed. In other embodiments, at least a portion of the intermediate acrylate product stream may be further treated after the alkylenating agent split.

As one example, the crude acrylate stream may be treated to remove light ends therefrom. This treatment may occur either before or after the alkylenating agent split, preferably before the alkylenating agent split. In some of these cases, the further treatment of the intermediate acrylate product stream may result in derivative streams that may be considered to be additional purified acrylate product streams. In other embodiments, the further treatment of the intermediate acrylate product stream results in at least one purified acrylate product stream.

In one embodiment, the inventive process operates at a high process efficiency. For example, the process efficiency may be at least 10%, e.g., at least 20% or at least 35%. In one embodiment, the process efficiency is calculated based on the flows of reactants into the reaction zone. The process efficiency may be calculated by the following formula.

Process Efficiency=2N_{HAcA}/[N_{HOAc}+N_{HCHO}+N_{H2O}]

where:

N_{HAcA} is the molar production rate of acrylate products; and

N_{HOAc}, N_{HCHO}, and N_{H2O} are the molar feed rates of acetic acid, formaldehyde, and water.

In other embodiments, the intermediate acrylate product stream comprises higher amounts of alkylenating agent. For example, the intermediate acrylate product stream may comprise from 1 wt. % to 10 wt. % alkylenating agent, e.g., from 1 wt. % to 8 wt. % or from 2 wt. % to 5 wt. %. In one embodiment, the intermediate acrylate product stream comprises greater than 1 wt. % alkylenating agent, e.g., greater than 5 wt. % or greater than 10 wt. %.

As mentioned above, the crude acrylate product of the present invention comprises little, if any, furfural and/or acrolein. As such the derivative stream(s) of the crude acrylate products will comprise little, if any, furfural and/or acrolein. In one embodiment, the derivative stream(s), e.g., the streams of the separation zone, comprises less than less than 500 wppm acrolein, e.g., less than 100 wppm, less than 50 wppm, or less than 10 wppm. In one embodiment, the derivative stream(s) comprises less than less than 500 wppm furfural, e.g., less than 100 wppm, less than 50 wppm, or less than 10 wppm.

### EXAMPLES

### Example 1

An example of a synthesized crude acrylate product was prepared by combining approximately 100 grams of acetic acid and approximately 100 grams of acrylic acid. The samples were kept at room temperature and pressure. By-product formation was measured over time as show in Table 3.

TABLE 3
By-product Formation Over Time
(Room Temperature and Pressure)

| | 3-acetoxypropanoic acid, |
|---|---|
| Time | wt % |
| Initial | 0 |
| 29 days | 0.41% |
| 43 days | 0.49% |
| 91 days | 0.55% |
| 107 days | 0.65% |

Table 3 shows that, even at room temperature and pressure, acetic acid and acrylic acid can be reacted to form at least a small amount of the Michael addition by-product, e.g., 3-acetoxypropanoic acid and/or 3-acetoxypropionic acid. This reaction drives down the overall efficiency of the acrylate product production process. Separation of the Michael addition by-product allows some of these reductions in efficiency to be re-captured, e.g., via re-use or recycle.

### Example 2

Additional examples of synthesized crude acrylate product were prepared in a manner similar to that discussed in Example 1. The samples were subjected to separation in a system as shown in FIG. 2. For multiple runs, by-product formation was measured after six hours in one column, e.g., the first column, of the separation zone. The results are shown in Table 4.

TABLE 4
By-product Formation Over Time
(Separation Zone)

| | 3-acetoxypropanoic acid, |
|---|---|
| Run | wt % |
| 1 | 1.00% |
| 2 | 1.02% |
| 3 | 1.08% |
| 4 | 1.21% |
| 5 | 1.07% |
| 6 | 0.99% |
| 7 | 1.30% |
| 8 | 1.41% |
| 9 | 1.16% |
| 10 | 1.28% |
| 11 | 1.58% |
| 12 | 1.77% |
| 13 | 1.89% |
| 14 | 1.56% |
| 15 | 1.85% |
| 16 | 1.97% |
| 17 | 2.01% |
| 18 | 2.64% |
| 19 | 2.66% |
| 20 | 1.50% |
| 21 | 1.82% |
| 22 | 1.98% |
| 23 | 1.92% |
| AVG. | 1.59% |

Table 4 shows that higher amounts of Michael addition product are formed when conditions are changed, e.g., when temperature and/or pressure are increased, as is the case in units of a separation zone. Thus, when a crude reaction mixture is subjected to separation, amounts, e.g., higher amounts, of Michael addition product is formed, as compared to room temperature combination. This formation drives down the overall efficiency of the acrylate product production process. Separation and subsequent recycling and/or re-use of the Michael addition by-product provides for improvement in reaction efficiency.

Because the process described herein accounts for the formed Michael addition product and subsequently treats same, additional acrylate product, e.g., acrylic acid, is formed from a similar amount of acetic acid, as compared to a conventional system, i.e., overall higher acetic acid conversion to acrylate product is achieved.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

1. A process for producing an acrylate product, the process comprising the steps of:
(a) reacting a reaction mixture comprising:
an alkanoic acid, and
an alkylenating agent,
under conditions effective to form a crude acrylate product comprising acrylate product and acetic acid;
(b) reacting at least a portion of the acrylate product and at least a portion of the acetic acid to form an intermediate acrylate product comprising a Michael addition by-product; and
(c) separating at least a portion of the intermediate acrylate product to form a by-product stream comprising Michael addition by-product and a purified acrylate product stream comprising acrylate product.

2. The process of claim 1, wherein step (a) is conducted in a reaction zone and step (b) is conducted in a separation zone.

3. The process of claim 2, further comprising the step of:
directing at least a portion of the by-product stream to the reaction zone.

4. The process of claim 3, wherein the reaction zone comprises a reactor comprising a decomposition catalyst.

5. The process of claim 4, further comprising the step of:
reacting at least a portion of the Michael addition by-product in the by-product stream to form acrylate product and alkanoic acid.

6. The process of claim 4, wherein the decomposition catalyst is selected from the group consisting of acid catalysts, heteropolyacids, gel ion exchange resins, sulfonated resins, sulfated and phosphated metal oxides, supported heteropolyacids, metal sulfates, metal phosphates, aluminosilcates, zeolites, mixed metal oxides, and mixtures thereof.

7. The process of claim 4, wherein the decomposition catalyst comprises sulfuric acid.

8. The process of claim 2, wherein the separation zone comprises at least one column and wherein step (b) is conducted in the at least one column.

9. The process of claim 8, wherein the separation zone comprises a first column and a second column and wherein step (b) is conducted in the first column.

10. The process of claim 9, wherein the by-product stream exits the first column.

11. The process of claim 10, further comprising the step of:
directing at least a portion of the by-product stream to the reaction zone.

12. The process of claim 11, further comprising the step of:
reacting at least a portion of the Michael addition by-product in the by-product stream to form recovered acrylate product and recovered alkanoic acid.

13. The process of claim 12, further comprising the step of:
directing at least a portion of the recovered acrylate product and alkanoic acid to the second column.

14. The process of claim 12, further comprising the step of:
directing at least a portion of the recovered acrylate product and alkanoic acid to a separation unit other than the first column.

15. The process of claim 12, further comprising the step of:
directing at least a portion of the recovered acrylic acid and acetic acid to the first column.

16. The process of claim 1, wherein step (b) is conducted at a temperature greater than 50° C.

17. The process of claim 1, wherein the Michael-addition by-product comprises 3-acetoxypropanoic acid and/or 3-acetoxypropionic acid.

18. The process of claim 1, wherein the purified acrylate product stream comprises less than 25 wt % Michael addition by-product.

19. The process of claim 1, wherein the by-product stream comprises at least 0.001 wt % Michael addition product.

20. A process for producing an acrylate product composition comprising acrylate product and Michael addition product comprising:
reacting alkylenating agent and alkanoic acid to form a crude acrylate product
heating the crude acrylate product to form the acrylate product composition comprising Michael addition product.
